(19) United States
(12) Patent Application Publication
(10) Pub. No.: US 2014/0228822 A1
(43) Pub. Date: Aug. 14, 2014

(54) COVER DEVICE FOR COVERING AN AT LEAST PARTLY RELEASED MEDICAL IMPLANT IN A CATHETER, AND CATHETER COMPRISING A COVER DEVICE, AND ALSO METHOD FOR COVERING AN AT LEAST PARTLY RELEASED MEDICAL IMPLANT WITH SUCH A COVER DEVICE

(71) Applicant: BIOTRONIK AG, Buelach (CH)

(72) Inventors: Biljana Trajkovic, Dielsdorf (CH); Dragica Pantic, Zuerich (CH); Amir Fargahi, Buelach (CH)

(73) Assignee: BIOTRONIK AG, Buelach (CH)

(21) Appl. No.: 14/168,704

(22) Filed: Jan. 30, 2014

Related U.S. Application Data
(60) Provisional application No. 61/763,973, filed on Feb. 13, 2013.

(51) Int. Cl. A61B 19/02

(52) U.S. Cl. CPC A61B 19/02
USPC 606/1

(57) ABSTRACT

A cover device (100), an insertion device (120), and a method for covering an at least partly released medical implant (102), which can be released from an insertion device (120) by a relative movement between a first and a second insertion element (52; 54), the cover device comprising at least one expansion body (10), at which a first radial extension (16) and at least one second radial extension (18) can be set, wherein the second radial extension (18) of the at least one expansion body (10) can be adapted to at least one radial extension (108) of the at least partly released medical implant (102) when the at least partly released medical implant (102) is covered, wherein the at least one expansion body (10) can be expanded substantially homogeneously in the circumferential direction (22) at least in at least one axial portion (20).

### CROSS REFERENCE TO RELATED APPLICATIONS

This application claims benefit of priority to U.S. provisional patent application Ser. No. 61/763,973 filed Feb. 13, 2013; the contents of which are herein incorporate by reference.

### TECHNICAL FIELD

The invention relates to a cover device for covering an at least partly released medical implant in a catheter and to a catheter comprising a cover device for covering an at least partly released medical implant during implantation in an animal and/or human body and also to a method for covering an at least partly released medical implant with such a cover device according to the preambles of the independent patent claims.

### BACKGROUND

In the field of medicine, implants are often used, which are introduced into an animal and/or human body either permanently or at least for a relatively long period of time in order to carry out replacement functions. For example, these implants include heart pacemakers, brain pacemakers for Parkinson's patients, cardiac implants, cochlear implants, retinal implants, dental implants, implants for joint replacement, vessel prostheses, or stents. In the field of cardiac implants, valve implants are known for example, such as aortic valve implants, which perform the function of the natural aortic valve. In this case, the valve implant is fixed immediately after implantation following expansion of the implant structure and adopts the position of the natural aortic valve.

Implants are connected to catheters before insertion into the body and have to be fastened such that they can be placed precisely and released in a defined manner by the catheter at the site of use without complication. A frequent problem here is that the implant is fixed with an incorrect position, which may lead to failure of the implant. To this end, it is known for example to slide an outer shaft of the catheter back over a partly released implant, whereby the implant is compressed into its original diameter. The implant is then implanted again.

One object of the invention is to specify a cover device, with which an implant with an insertion device can be covered in a simple and user-friendly manner and with which an implant can be released in a highly precise and targeted manner, for example by repositioning an implant.

A further object is to provide a corresponding insertion device.

A further object can be considered that of providing a method for covering the partly released medical implant with the aid of the cover device according to the invention, with which the medical implant can be inserted, covered, repositioned and released quickly, reliably and without complication.

### SUMMARY

The object is achieved in accordance with the invention by the features in the independent claims. Favorable embodiments and advantages of the invention will emerge from the other claims and the description.

A cover device for covering an at least partly released medical implant, which can be released from an insertion device by a relative movement between a first and a second insertion element, is proposed and includes at least one expansion body, at which a first radial extension and at least one second radial extension can be set, wherein the second radial extension of the at least one expansion body can be adapted to at least one radial extension of the at least partly released medical implant when the at least partly released medical implant is covered, wherein the at least one expansion body can be expanded substantially homogeneously in the circumferential direction at least in at least one axial portion.

Due to the embodiment according to the invention, a cover device can be provided, with which the at least partly released implant (referred to hereinafter as the released implant) can be covered quickly, without complication and gently. The duration of an intervention on a patient can thus also be reduced advantageously for the patient. An incorrectly placed, released implant can particularly advantageously be covered again or “resheathed” and therefore repositioned by means of the embodiments according to the invention. This is particularly the case since the at least one expansion body (also referred to hereinafter merely as the expansion body) can adapt flexibly to a contour of the implant. In addition, a functional test of the implant and, in the event of a defect, a removal of the dysfunctional implant can be assisted comfortably by the expansion body. Furthermore, with use of such an expansion body, an insertion device, such as a catheter, which is particularly compact can be used. In spite of an embodiment of the expansion body with a second extension, which is adapted to an extension of the released implant, a small, in particular radial extension of the insertion elements of the insertion device can thus be implemented. Compared to prior art systems, the insertion device therefore advantageously has a smaller diameter. In addition, a risk of deformation of the implant and of a resultant blocking of the insertion device can be eliminated by the use of the expansion body. As a further advantage, the concept of the expansion body can be applied to many different insertion devices and implants.

In this context, an implant is to be understood to mean a body that performs at least one replacement function permanently or for a relatively long period of time upon implantation in an animal and/or human body. Any medical implant appearing appropriate to a person skilled in the art would be conceivable here, such as a cardiac pacemaker, a brain pacemaker, a cardiac implant, a cochlear implant, a retinal implant, a dental implant, an implant for joint replacement, a vessel prosthesis or an appendix prosthesis, or a design of the medical implant as a valve implant is particularly advantageously proposed. In particular, a “valve implant” is to be understood in particular to mean a body that performs at least one replacement function of a check valve, either permanently or for a relatively long period of time upon implantation. Any medical valve implants appearing appropriate to a person skilled in the art would be conceivable here, such as an aortic valve implant, a pulmonary valve implant, a mitral valve implant or a tricuspid valve implant.

Here, an expansion body is to be understood to mean a body that can adapt passively and/or actively to a shape, for example the shape of another body, for example a region of an implant. In this case, adaptation is to be understood to mean an expansion or an enlargement as well as an action acting oppositely to the expansion or in other words a reduction. Here, the adaptation can be triggered by the expansion body itself or from outside the expansion body, for example by a change to a pressure, a temperature, a pH value, a concentration of a protein, of an ion, of a salt, etc. or by application of a voltage. Here, the radial extensions constitute diameters for example. The first extension is preferably smaller than the second extension. In addition, a multiplicity of different radial extensions can be set at the expansion body. Furthermore, the first and/or the second radial extension can be set merely at a region of the expansion body. The phrase “the second radial extension of the at least on expansion body [. . . is] adaptable to at least one radial extension of the at least partly released medical implant” is to be understood to mean that the second radial extension adapts substantially to the extension of an expanded region of the implant, wherein “substantially” means that a deviation of the extensions of up to 30% is considered as adapted.

Furthermore, in this context “substantially homogeneously expandable in the circumferential direction at least in at least one axial portion” is to be understood to mean that at least one region, which extends in the axial direction or in the longitudinal direction of the expansion body, expands substantially uniformly over its entire circumference. In other words, stated by way of example for a round embodiment of the expansion body, a change to the radial spacing of points arranged along the circumference of the expansion body at the same axial height from a central axis of the expansion body is substantially the same for all points. Here, substantially is also to be understood to mean deviations of the expansions or the expansion paths of the points of up to 10%.

In a preferred embodiment of the cover device, the at least one expansion body, at least once the second radial extension has been set, has a restoring force, which acts relative to an inner axis of the insertion device and is designed to trigger a reversal of the expansion of the at least partly released medical implant. The reversal of the expansion of the implant can thus be implemented in a constructionally simple manner on account of a force present in the expansion body, whereby space, including installation space, can be saved since a separate restoring means can be omitted. For example, the restoring force can be a radial force and/or a spring force. Alternatively and/or additionally, the expansion body may also be formed resiliently in regions. The portion of the expansion body at which the at least second radial extension is set conveniently has the restoring force acting relative to the inner axis of the insertion device.

A cover device that can be used in a particularly flexible and versatile manner can be provided if the at least second radial extension of the at least one expansion body is preferably twice as large, advantageously three times as large, and particularly preferably more than five times as large, as the first radial extension of the at least one expansion body. It has been found for example that embodiments of the expansion body can be selected, with which the first radial extension is 4 mm for example and the at least second radial extension is 25 mm for example. This thus gives an increase of the extension that is 6.25 times greater, or in other words a rise by 525%.

It may additionally be advantageous if the at least one expansion body has at least one braid, whereby the expansion body can be adapted particularly easily and flexibly to the radial extension of the implant. Furthermore, the expansion body can thus retain and/or adopt a smooth and regular shape, in particular in a curved state, as when the insertion device is passed along an aortic arch. The expansion body therefore has no hooks, sharp pointed edges and/or slits. The risk of injury of tissues and/or organs passed by the expansion body is therefore reduced and/or overcome in a patient-friendly manner. The expansion body therefore has at least one “braid” and/or is formed as a braid. In this case, a braid is to be understood to mean a structure having a plurality of strands made of ductile material knotted in one another. This material can be formed by any material considered usable by a person skilled in the art, such as a soft metal, a flexible plastics material and/or a textile material. In addition, a combination of a plurality of materials would also be conceivable. This could be a mixed material, or individual strands could be fabricated from different materials. The at least one expansion body may advantageously be fabricated from Elgiloy, whereby the expansion body is resistant to corrosion, in particular to bodily fluids, and has high strength, ductility and good endurance.

In a preferred embodiment the braid is reversibly collapsible so as to set the first radial extension and the at least second radial extension. Here, “reversibly collapsible” is to be understood to mean that the braid can assume at least two states (first and second radial extension), between which it can change due to a collapsing process and/or folding process, such as folding out and/or folding in. As a result of this embodiment, the changes in state can occur easily and quickly. In addition, a considerable change to the radial extension of the expansion body can thus be produced in a constructionally simple manner. In a further embodiment of the invention it is proposed that the at least one braid can include at least one metal braid and/or can be formed as a metal braid. An expansion body or braid designed in this way has very good sliding and guiding properties, whereby it can be manipulated in a particularly comfortable manner.

The at least one braid can advantageously and preferably be fabricated from intertwined round wire and the collapsing process can therefore be carried out advantageously with low friction. In addition, the expansion body can thus be equipped with a particularly homogeneous and gently acting surface and/or contour. Here, the round wire may be an individual wire, or a plurality of wires can be interwoven. In an alternative embodiment of the cover device, the at least one braid is fabricated from interwoven flat wire, whereby a wall thickness of the expansion body can be particularly thin. In general, a combination of round wire and flat wire would also be conceivable. The positive properties of these two embodiments can therefore advantageously be combined.

In the assembled state of the at least one expansion body in the insertion device, the expansion body includes a proximal end, which is remote from a distal end of the insertion device during use, and a distal end, which faces the distal end of the insertion device during use. In this case, the state of “use” represents the assembled state or the state of the implant with the insertion device during application or implantation. In an advantageous embodiment the at least one expansion body can be arranged at the distal end in a distal insertion device (see below) during use, whereby the released implant can be covered in a constructionally simple manner.

During use in a distal insertion device (see below), at least one grip segment may preferably be arranged at the proximal end of the at least one expansion body. The grip segment is designed to move the cover device and the expansion body independently of the insertion elements. A movement of the expansion body, in particular relative to one of the insertion elements or the outer insertion element, can thus be transferred reliably to the insertion element. The grip segment is therefore arranged on a part of any cover device located outside the body during the implantation process. The grip segment can be formed by a housing portion of the cover device and/or by a separate component arranged or integrally molded on the cover device. Good handling properties and a constructionally favorable design are provided if the grip segment of the cover device is arranged distally from at least one grip segment of one of the insertion elements or at least the outer insertion element.

During use, the at least one expansion body advantageously includes a proximal end, which is remote from a distal end of the insertion device during use, and a distal end, which faces the distal end of the insertion device during use. In accordance with an advantageous embodiment, at least the distal end of the at least one expansion body may be tapered and/or in particular may be formed with closed meshes. Contact between the distal end of the expansion body and vessel walls for example can thus be designed so as to be gentle, causing little irritation. Closed meshes are to be understood here as connected wires.

The at least one expansion body preferably has a coating at least in a partial region. The expansion body can thus be designed and/or adapted particularly for specific functions. This coating can be applied for example to a radial inner surface, against which at least part of the implant bears radially in the covered state, and/or can assist a sliding movement of the expansion body relative to the partly released implant during the covering process. An expansion body that can be used in a versatile manner can be provided if the coating is formed by a plastics film. Such a coating can be applied in a constructionally simple manner to the partial region, for example by a spraying or dipping method. In particular, this plastics film is very thin, wherein very thin is to be understood to mean less than 0.5 mm. The plastics film is preferably between 0.3 mm and 0.1 mm, in particular 0.2 mm thick.

The proximal end and the distal end of the cover device can preferably be fabricated from different materials, whereby properties such as strength, resilience, pliability, slidability, for example over a coating that increases or reduces friction, etc. can be matched individually to the requirements of the portion of the cover device. With use of the cover device in a distal insertion device, the distal end preferably constitutes the expansion body itself.

With the embodiment of the cover device as a stabilizing tube of the insertion device, a gentle and low-friction feed of the distal end of the insertion device with the implant can be enabled. In addition, the cover device fulfills two functions, namely that of covering the implant or covering the implant again and that of feeding the distal end of the insertion device in a stable manner, whereby installation space, component parts, assembly effort and costs can be saved advantageously.

In accordance with a further aspect of the invention, an insertion device for inserting a medical implant, which can be released by a relative movement between a first and a second insertion element, is proposed and includes a cover device for covering a partly released medical implant, the cover device including at least one expansion body, at which a first radial extension and at least one second radial extension can be set, wherein the second radial extension of the at least one expansion body can be adapted to at least one radial extension of the at least partly released medical implant when the at least partly released medical implant is covered, wherein the at least one expansion body can be expanded substantially homogeneously in the circumferential direction at least in at least one axial portion.

As a result of the embodiment according to the invention, an insertion device can be provided, with which the at least partly released implant can be covered quickly, without complication and gently. The duration of an intervention on a patient can thus also be reduced advantageously for the patient. In addition, on account of a resheathing, the incorrectly placed released implant can be repositioned due to the flexible adaptation of the expansion body to the contour of the implant. Furthermore, a functional test of the implant can be carried out and, in the event of a defect, the dysfunctional implant can be removed in a comfortable manner assisted by the expansion body. An insertion device may also be formed which is of particularly compact design. Compared to prior art systems, the insertion device therefore advantageously has a smaller diameter. In addition, a risk of deformation of the implant and of a resultant blocking of the insertion device can be eliminated by such an insertion device. A further advantage is that the concept of the expansion body can be applied to many different insertion devices and implants.

The insertion device can be designed in two variants for example. These variants differ in terms of the end at which the release of the implant begins. This can occur either at the distal end, which is arranged in the direction of the distal end of the insertion device, or at the proximal end, which is arranged in the direction of the proximal end of the insertion device. In this case, each insertion device is moved in a direction of insertion. In this context, the direction of insertion is to be understood to mean the direction along which the insertion device with the cover device and the implant is inserted into the human and/or animal body. In particular, this direction points from the proximal end of the insertion device to the distal end of the insertion device.

If the release of the implant starts at the distal end thereof, the cover device can be used for what is known as a distal insertion device. In this case, the inner insertion element (first insertion element) is connected to a tip of the insertion device, such as a catheter tip. The outer insertion element (second insertion element), which is arranged radially around the inner insertion element, is by contrast not connected to the catheter tip and can be moved relative to the catheter tip by an axial movement in the direction of the proximal end of the insertion device. The distal end of an implant arranged radially between the inner and outer insertion element may thus be exposed and/or released first for implantation or expansion of the implant.

If the cover device is designed in accordance with the alternative embodiment, with which the release of the implant starts at the proximal end thereof, the cover device can be used for what is known as a proximal insertion device. In this case, the outer insertion element is connected to the tip of the insertion device, but by contrast the inner insertion element is not. To move the outer insertion element, the outer insertion element is coupled to a guide element. The guide element runs coaxially with the inner insertion element and therein and for example is formed by a shaft having an insertion wire and a lumen. For manipulation by an operator, the guide element is connected to the proximal end of the insertion device. The guide element is also connected to the tip. If the insertion element is then displaced in the direction of the distal end of the insertion device, it pushes both the tip and the outer insertion element into the distal direction, whereby an opening and/or a gap is formed and the proximal end of an implant arranged radially between the inner and outer insertion element is exposed or released first for implantation or expansion of the implant. Here, the cover device would move in the direction of the proximal end of the insertion device and should be moved via a further guide element running radially within the inner insertion element.

The insertion device according to the alternative embodiment with the release of the proximal end of the implant first can be used particularly advantageously when implanting asymmetrical implants, for example in cardiac catheter applications.

The expansion body preferably extends in the circumferential direction around one of the insertion elements. It is additionally proposed for the at least one expansion body of the cover device to be placed radially around at least one of the insertion elements and in particular around the outer insertion element. As a result of this embodiment, the expansion body may advantageously be adapted to a shape, in particular a round shape, of the insertion element, which enables a space-saving connection between the expansion body and the insertion element. Furthermore, it may be advantageous if the at least one expansion body of the cover device is arranged axially movably with respect to at least one of the insertion elements and in particular preferably with respect to the outer insertion element. The released implant can thus be covered in a constructionally simple manner and without hindrance.

With the embodiment of the insertion device as a distal insertion device, the second or the outer insertion element may include a receptacle for the implant. Here, the implant preferably bears against a radial inner wall of the second insertion element, whereby the second insertion element may act as a clamping body for the implant. In this context a “clamping body” is understood to mean a body that holds another element, in particular the implant, in the insertion device in a fixed position by means of a clamping effect and/or a force-locked connection. The implant is thus held captively in the insertion device in a clamped state. In the clamped state, an interaction between an expansion force of the implant and a clamping force of the second insertion element for example holds the implant in position, whereby the implant is prevented from sliding out from the insertion device or the clamping body. The clamping body may additionally have a holding effect, for example as a result of a static friction, designed separately from the clamping effect. An embodiment of the clamping body with a (additional) specific material property would also be conceivable here.

The implant can thus be fixed in a spatially comfortable manner if the implant is fixed in a protective sleeve of the second insertion element. This protective sleeve has a greater radial diameter than the second insertion element and forms a functional part of the second insertion element, that is to say it can be moved together with the second insertion element. The protective sleeve is connected captively to the second insertion element. Here, any type of connection considered expedient by a person skilled in the art can be considered, such as a force-locked connection, an interlocking connection or an integrally bonded connection, for example by means of welding, soldering, screwing, nailing or adhesive bonding. With an embodiment of two connected component parts, properties such as size, material, coating, friction etc., can be matched individually to the requirements of the component part. In principle, the protective sleeve may also be formed in one piece with the second insertion element, wherein “in one piece” is to be understood to mean that the protective sleeve and the second insertion element are formed by the same component part and/or from a cast part and/or can only be separated from one another with a loss of function of at least one of the component parts. The protective sleeve may also have a greater radial extension than an inner diameter of the distal end of the at least one expansion body in its first radial extension. The insertion device can thus be formed with a practically constant extension from its proximal end to its distal end.

In accordance with an advantageous embodiment of the invention, the first or the inner insertion element is designed to hold the implant in position at least in the event of expansion of the implant. This can be achieved by means of any principle considered usable by a person skilled in the art, such as a force-locked connection and/or interlocking connection, a static friction or the like. A distal end region of the first insertion element may preferably be formed as an implant holder, which has at least one structure, such as a hook, an eyelet, a slit, etc., which is designed to interact with a contact structure of the implant. These are preferably formed as eyelets, which, in the assembled state of the implant, are arranged for example in the distal insertion device at the end of the implant pointing toward the proximal end of the insertion device.

In accordance with an advantageous embodiment, the implant can be designed as a self-expanding implant, whereby it can open automatically in the absence of the outer insertion element and/or upon exit therefrom. An additional expansion means can be omitted as a result of the self-expanding implant. Space and assembly effort for this can thus be saved advantageously. The insertion device can thus also be designed in a less complex manner. In principle, it would also be possible however to use a balloon-expandable implant. For this purpose, the insertion device would have to be adapted accordingly however, which can be achieved automatically by a person skilled in the art on the basis of his knowledge in the art.

In addition, a method for covering an at least partly released implant with a cover device of an insertion device is proposed. The method includes at least the following steps: performing a relative movement of at least one expansion body of the cover device with respect to the at least partly released implant and thus adapting at least one second radial extension of the at least one expansion body to at least one radial extension of the at least partly released medical implant, wherein the at least one expansion body is expanded substantially homogeneously in the circumferential direction at least in at least one axial portion.

As a result of the embodiment according to the invention, a method can be implemented, by means of which an at least partly released implant can be placed in position quickly, without complication and gently, and ultimately can be implanted correctly. The duration of an intervention on a patient can thus be reduced, in particular advantageously for the patient. An already partly released implant can particularly advantageously be withdrawn into at least one of the insertion elements and repositioned in a user-friendly manner by means of the method according to the invention. In addition, a functional test can be carried out on an implant thus released and the implant can be removed in the event of a defect. Furthermore, an insertion device that is particularly compact can be used with such a method. In addition, a risk of deformation of the implant and of a resultant blocking of the insertion device can be eliminated. A further advantage is that the method can be applied to many different insertion devices and implants. The method according to the invention for example is a transcatheter aortic valve implantation method (TAVI method) for a hybrid bioprosthesis.

The method according to the invention additionally includes the following step: reversal of the expansion of the at least partly released medical implant triggered by a restoring force, at least of the expansion body of the cover device, acting relative to an inner axis of the insertion device. The reversal of the expansion of the implant can thus be implemented in a constructionally simple manner on account of a force provided in the expansion body, whereby a simple mechanism can be applied.

### DESCRIPTION OF THE DRAWINGS

The invention will be explained in greater detail hereinafter by way of example on the basis of an exemplary embodiment illustrated in the drawings, in which:

FIG. 1 shows a schematic illustration of a section through a preferred and convenient exemplary embodiment of a distal insertion device and a cover device according to the invention with an expansion body and an implant;

FIG. 2 shows a schematic illustration of the expansion body in FIG. 1 in a state with a first radial extension;

FIG. 3 shows a schematic illustration of the expansion body in FIG. 1 with a set second radial extension at one end;

FIG. 4 shows a schematic illustration of an end of the expansion body in FIG. 1 with closed meshes at one end;

FIG. 5 shows a schematic illustration of two crossing portions of a round wire of the expansion body in FIG. 1;

FIG. 6 shows a schematic illustration of a mesh of the expansion body in FIG. 1;

FIG. 7 shows a schematic illustration of the mesh of the expansion body in FIG. 6 in an unexpanded state (A) and in an expanded state (B);

FIG. 8 shows a schematic illustration of the expansion body in FIG. 1 in its unexpanded, straight state;

FIG. 9 shows a schematic illustration of the expansion body in FIG. 8 in a curved state;

FIG. 10 shows a schematic illustration of the insertion device and the cover body from FIG. 1 with a fully placed implant before a release of the implant;

FIG. 11 shows a schematic illustration of the insertion device and the cover body from FIG. 1 with a partly released implant;

FIG. 12 shows a schematic illustration of the insertion device and the cover body from FIG. 1 with the partly released implant covered by the expansion body in FIG. 3;

FIG. 13 shows a schematic illustration of the insertion device and the cover body from FIG. 1 with a resheathed implant;

FIG. 14 shows a schematic illustration of the insertion device and the cover body to from FIG. 1 with an implant before renewed release thereof;

FIG. 15 shows a schematic illustration of the insertion device and the cover body from FIG. 1 with the partly released implant covered by the expansion body in FIG. 3 with a blockage during a sheathing process;

FIG. 16 shows an illustration of the method steps of an insertion method and covering method according to the invention on the basis of a block diagram;

FIG. 17 shows a schematic illustration of the insertion device and the cover body from FIG. 11 with a partly released implant during implantation at a site of implantation, and;

FIG. 18 shows a schematic illustration of the insertion device and the cover body from FIG. 17 with a partly released implant once the implant has been covered with the expansion body in FIG. 3.

### DETAILED DESCRIPTION

In the figures, functionally like or similarly acting elements are denoted in each case by like reference signs. The figures are schematic illustrations of the invention. They do not show specific parameters of the invention. The figures also merely reproduce typical embodiments of the invention and are not intended to limit the invention to the embodiments illustrated.

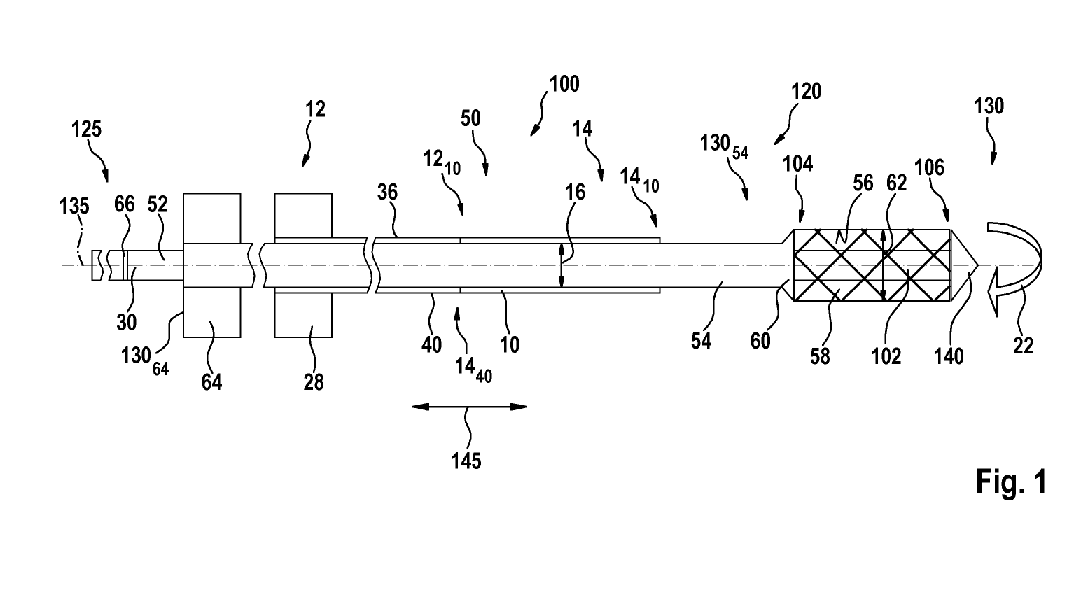

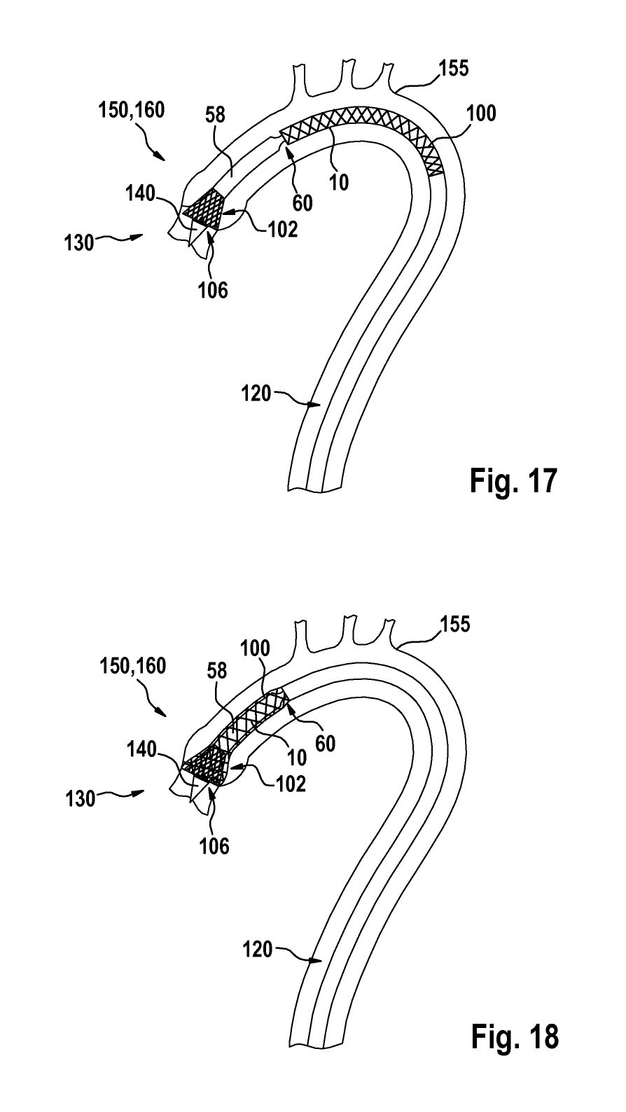

FIG. 1 shows a longitudinal section through a preferred exemplary embodiment of a cover device 100 of a distal insertion device 120 for inserting a medical self-expanding implant 102 in the form of a folding implant or a stent having a folding structure (not shown in detail). For example, the insertion device 120 is a catheter having a shaft region 50 with two coaxially arranged insertion elements 52, 54, for example an inner shaft (insertion element 52) and an outer shaft (insertion element 54), which surrounds the inner shaft. The proximal end 125 of the insertion device 120 faces a user during use, that is to say as the implant 102 is fastened to the insertion device 120 or during implantation. The implant 102 is placed at a distal end 130 of the shaft region 50 between the inner shaft and the outer shaft and is to be released at the site of implantation 150, such as an aortic annulus, in the animal or human body (see FIGS. 17 and 18).

The implant 102 is arranged at the end 130 of the shaft region 50 remote from the user, for example in the vicinity of a catheter tip 140. For example, the implant 102 is placed around the inner insertion element 52 or is fixed axially to an implant holder (not shown in detail) of the inner insertion element 52 by fastening structures (not illustrated), such as eyelets, which are formed integrally on a proximal end 104 of the implant 102. The first insertion element 52 is thus designed to hold the implant 102 axially in position in the event of expansion of the implant. For radial fixing, the implant 102 bears against a radial inner surface 56 of a protective sleeve 58. This protective sleeve 58 is arranged or formed integrally on a distal end 13054 of the outer insertion element 54 via a connection region 60. The protective sleeve 58 has a greater radial extension 62 than the outer insertion element 54 and is adapted in terms of its dimensions to the implant 102, in particular in the unexpanded state of the implant. The implant 102 can be released by a relative movement between the first and the second insertion element 52, 54, starting at a distal end 106 of the implant 102. In this case, the inner insertion element 52 is connected to the catheter tip 140, but by contrast the outer insertion element 54 is not.

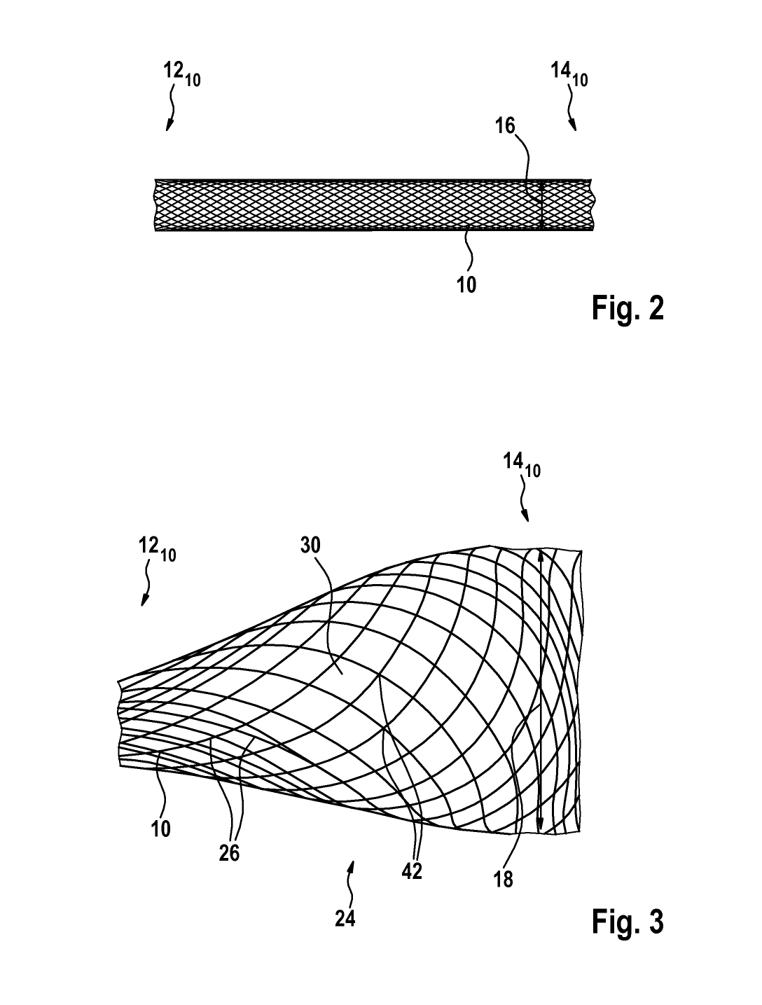

The cover device 100 is designed as a stabilizing tube 36 of the insertion device 120 and is placed radially around the outer insertion element 54. In addition, the cover device 100 is arranged axially movably with respect to both insertion elements 52, 54. The cover device 100 is used to cover the implant 102 partly released by the insertion device 120. To this end, the cover device 100 includes an expansion body 10, at which a first radial extension 16 and a second radial extension 18 can be set (see below and FIGS. 2 and 3). The cover device 100 also has a proximal end 12, which is remote from the distal end 130 of the insertion device 120 during use, and a distal end 14, which faces the distal end 130 of the insertion device 120 during use. The expansion body 10 is arranged at the distal end 14 of the cover device 100 and, during use of the insertion device 120, is placed proximally from the protective sleeve 58 holding the implant 102.

A grip segment 28 is arranged at the proximal end 12 of the cover device 10. This grip segment 28 is placed distally from grip segments 64 of the insertion elements 52, 54, which in turn are arranged at the proximal end 125 of the insertion device 120. Here, merely the grip segment 64 of the outer insertion element 54 is shown. All grip segments 28, 64 remain outside the body during the implantation process.

FIG. 2 shows the expansion body 10 in the state of its first radial extension 16, and FIG. 3 shows the expansion body 10 in the state of its second radial extension 18. This extension 16, 18 is in each case a diameter of the expansion body 10 or of a region thereof. The first radial extension 16 may be approximately 4.85 mm for example and the second radial extension 18 may be 20.83 mm for example. The second radial extension 18 is therefore approximately five times greater than the first radial extension 16, giving an expansion rate of approximately 430% (see below for calculation of the values).

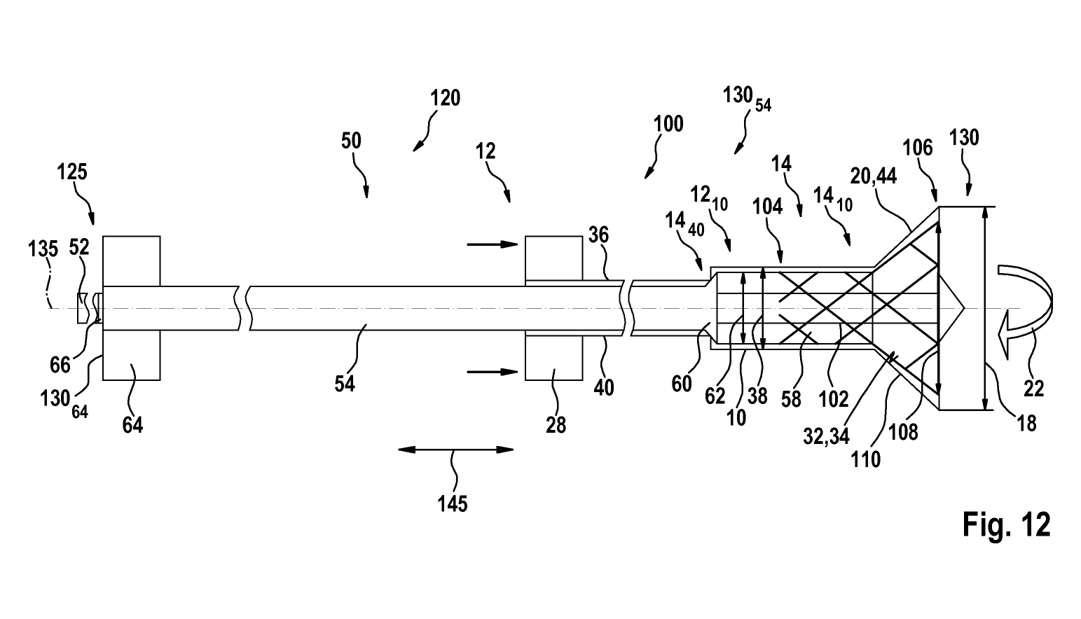

Here, the second radial extension 18 is set merely at one end 14₁₀ of the expansion body 10, wherein this end 14₁₀ faces the distal end 130 of the insertion device 120 during use in the insertion device 120 and thus forms the distal end 14₁₀ of the expansion body. The other end 12₁₀ or the end 12₁₀ that is remote from a distal end 130 of the insertion device 120 during use, that is to say forms the proximal end 12₁₀, can substantially retain the first radial extension 16 or can adopt a further radial extension 38 depending on the embodiment of the outer insertion element 54, as here with a protective sleeve 58 widened with respect to the outer insertion element 54 (see also FIG. 12 and see below). In the region between the proximal end 12₁₀ and the distal end 14₁₀, the radial extension increases substantially uniformly. This is also dependent however on an outer contour of the outer insertion element 54 and/or of the protective sleeve 58. The proximal end 14₁₀ of the expansion body 10 is connected to a shaft casing 40 of the proximal part 14 of the cover device 100. In this case, any connection method considered usable by a person skilled in the art can be considered, such as welding, soldering, screwing, nailing, knotting or adhesive bonding (see FIG. 1).

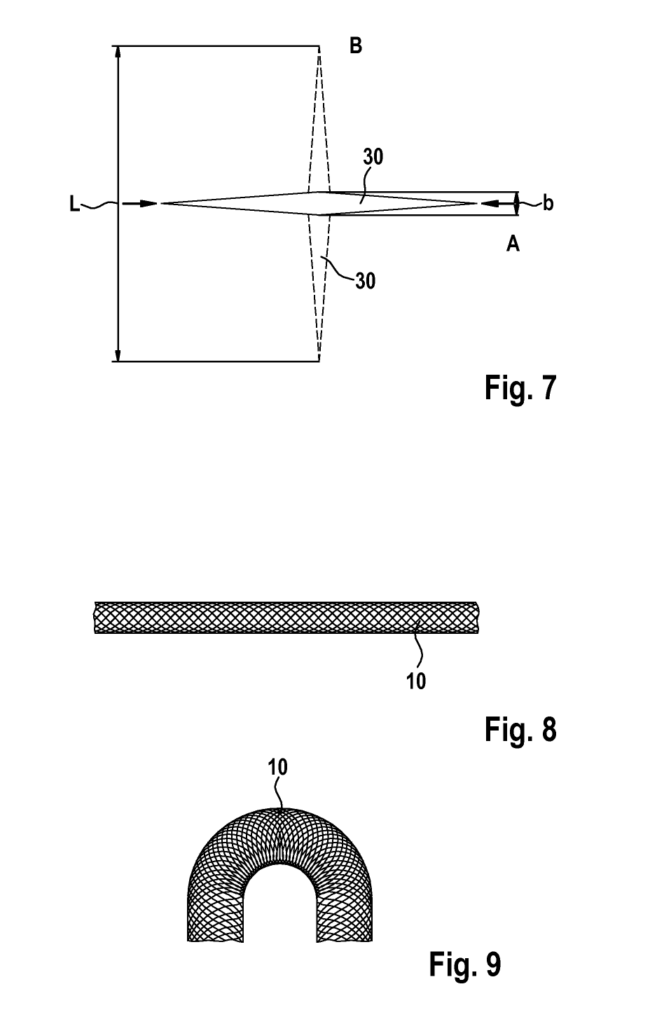

As can be seen in particular in FIG. 3, the expansion body 10 has a braid 24 or a metal braid, which is fabricated from interwoven round wire 26. This round wire 26 and therefore the expansion body 10 consists for example of Elgiloy. In addition, the braid 24 is reversibly collapsible in order to set the first radial extension 16 and the second radial extension 18 (see also FIG. 7).

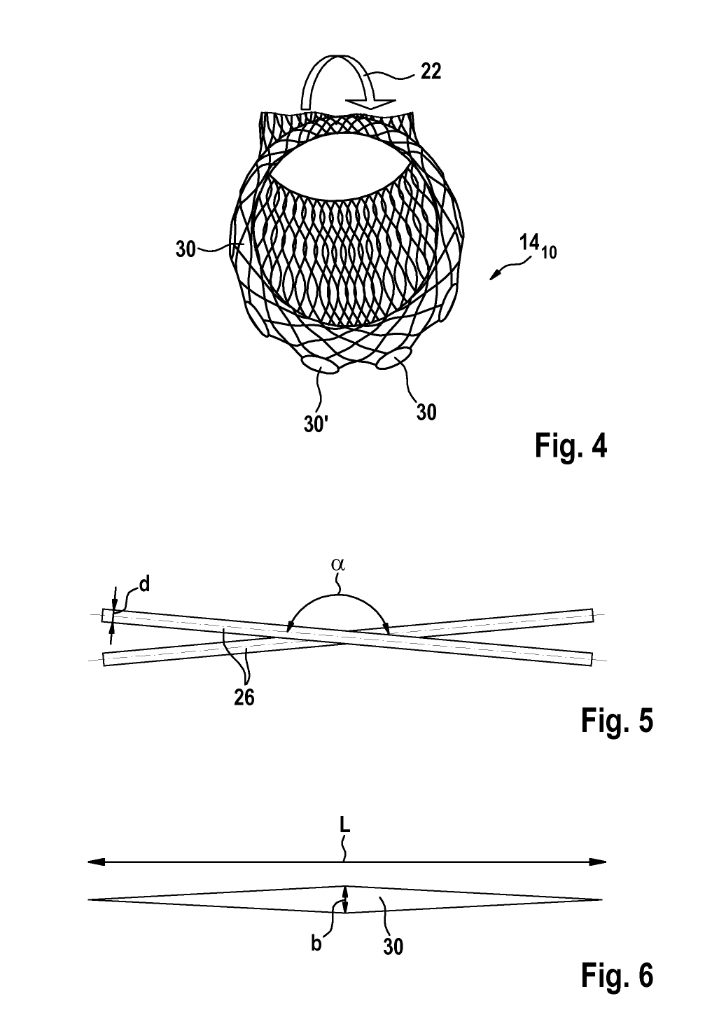

FIG. 4 shows a view of the distal end 1410 of the expansion body 10. In order to minimize a risk of injury to a patient during implantation and in particular during displacement of the cover device 100, the distal end 14₁₀ is formed in a tapered manner and with closed meshes 30, 30′. In order to match the cover device 100 well to its functions, the proximal end 12 and the distal end 14 are fabricated from different materials. As described above, the expansion body 10 arranged at the distal end 14 consists of Elgiloy. The proximal end 12 can be fabricated for example from a stable yet flexible plastics material. The grip segment 28 by contrast is preferably fabricated from a hard, dimensionally stable material.

The dimensions of the expansion body 10 and of its components are illustrated in FIGS. 5 to 7. A diameter d of the round wire 26 is 0.2 mm for example and an angle of crossing a, which is produced in the unexpanded state of the expansion body between the round wires 26, is 175° for example (see FIG. 5). An axial length L of a mesh 30 arranged between the ends 12₁₀ and 14₁₀ of the expansion body 10 is 5 mm for example (see FIGS. 3 and 6). A width b of the mesh 30 in the circumferential direction 22 of the expansion body 10 is 0.9 mm for example (see FIGS. 3, 4 and 6). If the expansion body 10 or the braid 24 then has for example 15 meshes 30 or crossing points 42 in the circumferential direction 22, a circumference of the expansion body 10 in the unexpanded state of 13.5 mm (15×the material thickness d of the round wire 24 of 0.2 mm+15×the width b of 0.9 mm, see FIGS. 3, 5 and 7A) and a circumference of the expansion body 10 in the expanded state of 78 mm (15×the material thickness d of the round wire 24 of 0.2 mm+15×the length L of 5 mm, see FIGS. 3, 5 and 7B) are given. The circumference of the expansion body 10 in the expanded state may thus assume a value approximately five times greater than the circumference of the expansion body 10 in the unexpanded state, giving an expansion rate of approximately 470% (the discrepancy with respect to the expansion rate disclosed previously is due to the fact that the inner diameters were compared above and the outer diameters are relevant here).

FIG. 8 shows a portion of the expansion body 10 in its unexpanded, straight state, as is provided in particular in an unassembled state on the insertion device 120. FIG. 9 shows this region of the expansion body 10 in a curved state, as may be present in particular when feeding the insertion device 120 to the site of implantation 150 via an aortic arch 155 for example (see also FIG. 17).

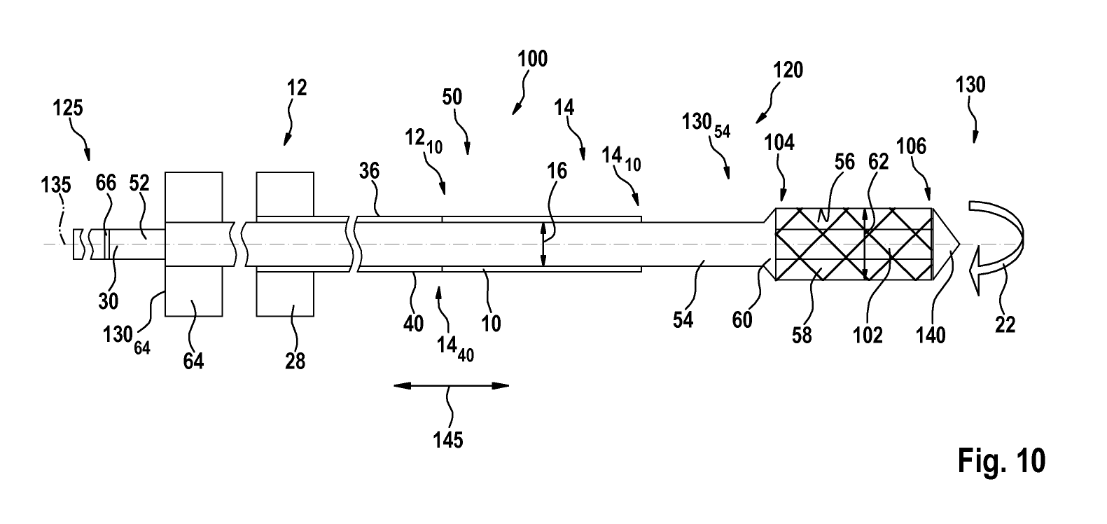

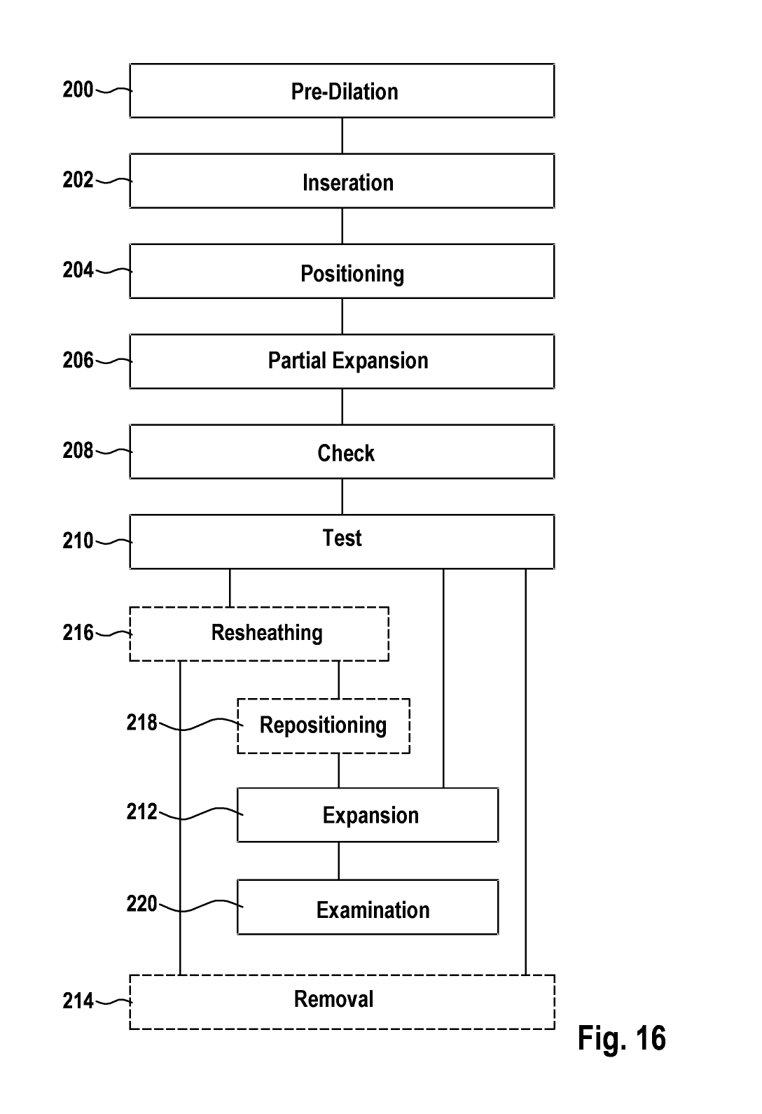

The insertion of the medical implant 102 with the aid of the insertion device 120, by way of example for the implantation of an artificial cardiac valve at an aortic annulus, and a method for covering the partly exposed implant 102 with the cover device 10 will be described hereinafter on the basis of FIGS. 10 to 15 and FIGS. 17 to 18 and on the basis of the block diagram in FIG. 16. The implant 102 is assembled in/on the insertion device 120 as described above and as shown in FIG. 10.

Before the insertion device 120 is inserted, the native aortic valve 160 is pre-dilated in step 200 (pre-dilation) using what is known as a valvuloplasty balloon. The insertion device 120 thus prepared is then inserted in step 202 (insertion) into the body in a known manner with the aid of a guide element (not shown in greater detail), that is to say with the aid of what is known as a guide wire, for implantation of the implant 102 in the body and is positioned in step 204 (positioning). In this case, a valve plane of the valve structure (not shown) should be arranged flush with the aortic annulus of the natural valve 160 (see FIGS. 16 and 17, natural valve not shown in detail).

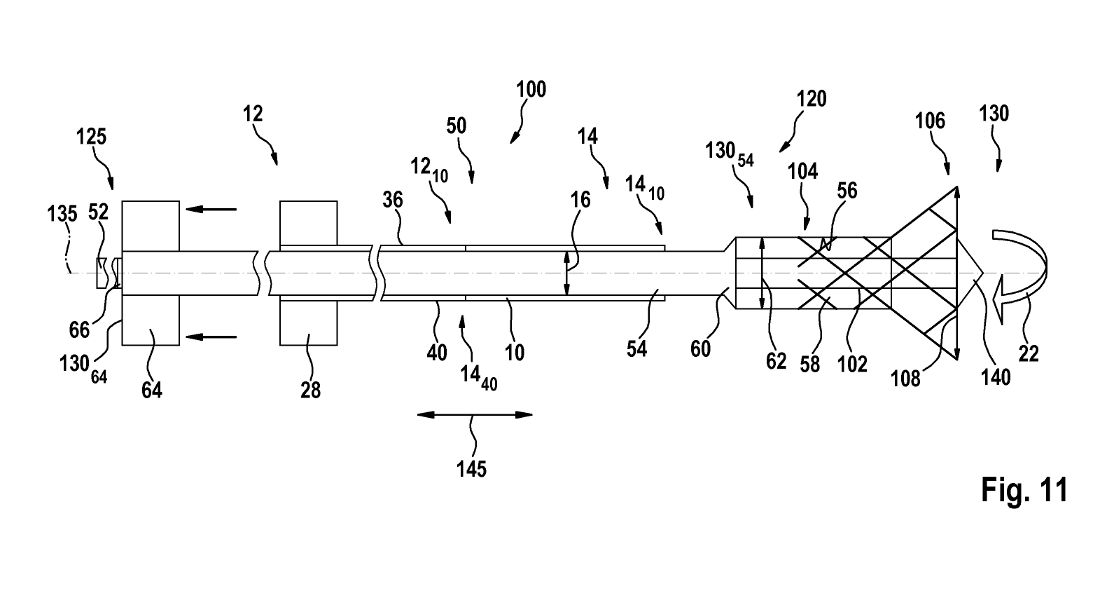

The implant 102 then starts to be placed in position. Here, the distal end 106 of the implant 102 is exposed in a first step 206 (partial expansion), whereby the implant expands automatically (see FIGS. 11 and 17). In this case, the second insertion element 54 is drawn via the grip segment 64 in the direction of the proximal end 125 of the insertion device 120 (see arrow in FIG. 11), whereby the second insertion element 54 performs a relative movement in the axial direction 145 with respect to the first insertion element 52 and the cover device 100. For this purpose, the first insertion element 52 and the cover device 100 may possibly have to be axially fixed at their grip segments 28, 64. The drawing movement is carried out in this case until a proximal end 130₆₄ of the grip segment 64 of the second insertion element 54 is located at an axial height of a stop 66 of the first insertion element 52 (see the axial heights of the grip segment 28 in FIGS. 10 and 11). The relative movement is thus limited on account of the stop 66 (see FIG. 11). This stop 66 constitutes a marker for example.

The implant 102 is thus partly exposed and the distal end 106 has adopted a radial extension 108 in its state thus expanded. Here, the radial extension 108 is minimally smaller than the second radial extension 18 of the expansion body 10, which is adopted by the expansion body when covering the partly exposed implants 102 (see below and FIG. 12).

At this point, a position check of the expanded distal end 106 of the implant 102 can be carried out in step 208 (check). In addition, a functional test of the implant 102 or the folding structure thereof can then be carried out in step 210 (test). If the distal end 106 of the implant 102 is located in the desired and correct position, as is symbolized in FIG. 16 by a tick, the proximal end 104 of the implant 102 is exposed in a step 212 (expansion) by withdrawing the second insertion element 54 further in the direction of the proximal end 125 of the insertion device 120 (not shown in detail, see FIG. 16). If a dysfunction is established, the implant 102 can be removed again from the body in step 214 (removal), as is symbolized in FIG. 16 by a cross (see FIG. 16). Since step 214 is an alternative method step, it is shown in FIG. 16 as a box with a dashed outline.

If an incorrect position of the distal end 106 is determined or if the folding structure functions unsatisfactorily in the position, this can be remedied in the next steps, as symbolized in FIG. 16 by a cross. In this case, the end 106, which is already expanded, of the implant 102 must first be covered again or “resheathed” in a step 216 (resheathing). For this purpose, the released end 106 of the implant 102 is first transferred back into its unexpanded state. In doing so, the expansion body 10 is slid over the already expanded distal end 106 of the implant 102 in order to cover the partly released implant 102 by implementing a relative movement of the expansion body 10 with respect to the at least partly released implant 102 and with respect to the second insertion element 54 in the axial direction 145, as is shown in FIGS. 12 and 18 (see arrow in FIG. 12).

The expansion body 10 is thus adapted, at least in its second radial extension 18, to the radial extension 108 of the partly released implant 102 or an axial portion 20, or, in this exemplary embodiment, a distal third 44 of the expansion body 10 adapts, at the distal end 14₁₀ thereof, to an outer contour 110 of the released distal end 106 of the implant 102. Here, the expansion body 10 is expanded homogeneously in the circumferential direction 22 and additionally in the axial direction 145 in an axial portion 20 or in its distal third 44.

The expansion body 10 expands like a funnel so to speak around the partly released implant 102. This can be implemented in a simple manner, since it has been found that a friction between the material of the implant 102, such as Nitinol, and the expansion body 10 is lower than a friction between the implant 102 (Nitinol) and an aortic wall and/or a stenosis at the site of implantation 150. To assist the relative movement between the expansion body 10 and the implant 102, a partial region 32, more specifically an inner surface in the distal third 44 of the expansion body 10, has a coating 34, which is formed by a thin plastics film. This plastics film consists of Teflon and has a thickness of 0.2 mm.

The movement of the cover device 100 is limited by contact between the distal end 14₄₀ of the shaft casing 40 and the conically outwardly running connection region 60 between the second insertion element 54 and the protective sleeve 58. In this case, the dimensions, in particular the lengths, of the component parts (shaft casing 40, expansion body 10, implant 102, protected sleeve 58, position of the stop 66) or the movement play thereof are matched to one another such that the distal end 14₁₀ of the expansion body 10 is positioned substantially at the same axial height as the distal end 106 of the implant 102 after the partial expansion. In addition, the proximal end 12₁₀ of the expansion body 10 also expands in the event of movement of the cover device 100. The expansion body adopts the further radial extension 38, which adapts to the radially widened protective sleeve 58.

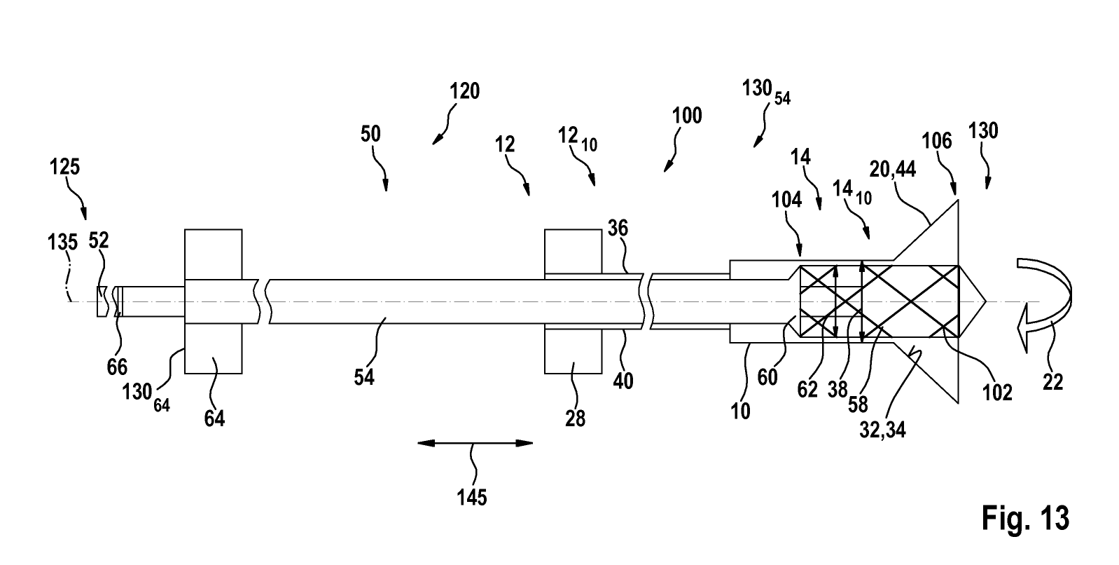

Since the expansion body 10 has a restoring force acting relative to an inner axis 135 of the insertion device 120 when the second radial extension 18 has been set and the restoring force is designed to trigger a reversal of the expansion of the partly released implant 102, the expansion of the partly released implant 102 is then reversed by pressing the expanded distal end 106 of the implant 102 radially inwardly and returning it into its unexpanded state (see FIG. 13).

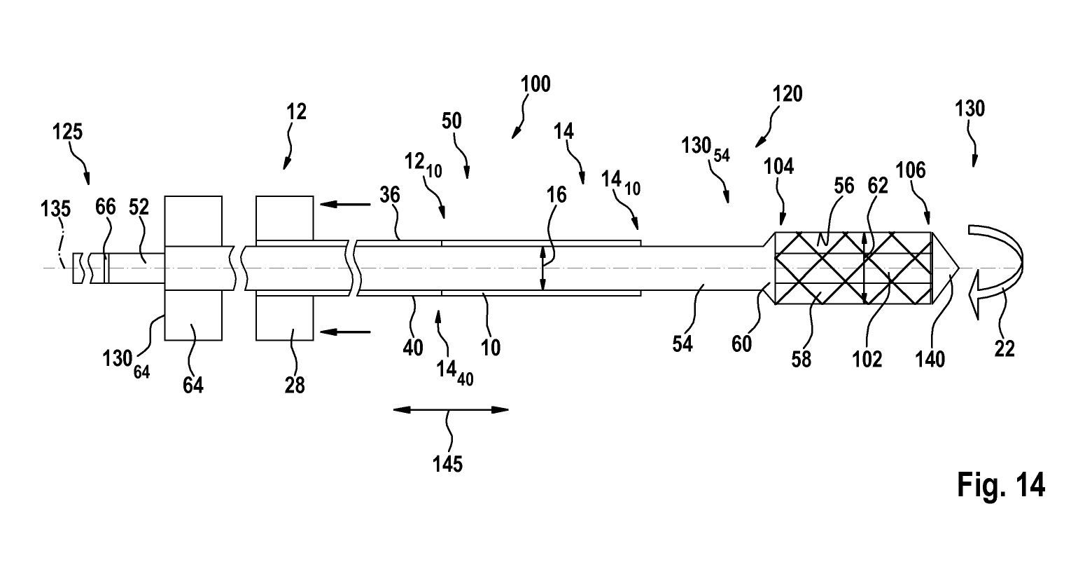

The actual resheathing process 216 can then take place. Here, the first insertion element 52 is drawn in the axial direction 145 toward the proximal end 125 of the insertion device 120 (see arrow in FIG. 13). The distal end 106 of the implant 102 previously compressed by the restoring force of the expansion body 10 thus reenters the protective sleeve 58 of the second insertion element 54. The distal third 44 of the expansion body 10 is shown in an expanded state in FIG. 13 in order to provide an improved illustration of the compressed implant 102 located again in the protective sleeve 58, although the distal third 44 here has assumed the further radial extension 38, adapted to the extension 62 of the protective sleeve 58. The cover device 100 is then drawn in the axial direction 145 toward the proximal end 125 of the insertion device 120 (see arrow in FIG. 14), whereby the expansion body 10 again adopts the first radial extension 16 and the cover device 100 is thus located again in its starting position (see FIG. 14).

The distal end 106 of the implant 102 can then be positioned again in step 218 (repositioning) by withdrawing again the second insertion element 54, as is symbolized in FIG. 16 by a tick (see FIG. 11). Since steps 216 and 218 are alternative method steps, they are shown in FIG. 16 as boxes with a dashed outline. If the distal end 106 of the implant 102 is then located in the desired and correct position, it can be finally released (symbolized by a tick in FIG. 16). For this purpose, the implant 102 is released from the implant holder. This is achieved for example by radially withdrawing hooks of the implant holder from the fastening eyelets at the proximal end 104 of the implant 102 (not shown). In a step 212 (expansion), the entire implant 102 can then be released (not shown in detail, see FIG. 16). In this case, the expansion of the proximal end 104 of the implant 102 is triggered by withdrawing the second insertion element 54 further in the direction of the proximal end 125 of the insertion device 120, whereby the implant expands automatically. Lastly, the insertion device 120 is withdrawn and removed from the body in step 214 (removal) (see FIG. 16). The implant 102 remains fully positioned in the body (not shown). In order to verify successful implantation, an aortogram can then be taken in step 220 (examination).

Alternatively, the second insertion element 54 could also be slid in the axial direction 145 toward the distal end 130 of the insertion device 130 during the actual resheathing process 216, whereby the implant 102 is again arranged completely in the protective sleeve 58. In this case, the drawing movement is carried out until the implant 102 is completely released.

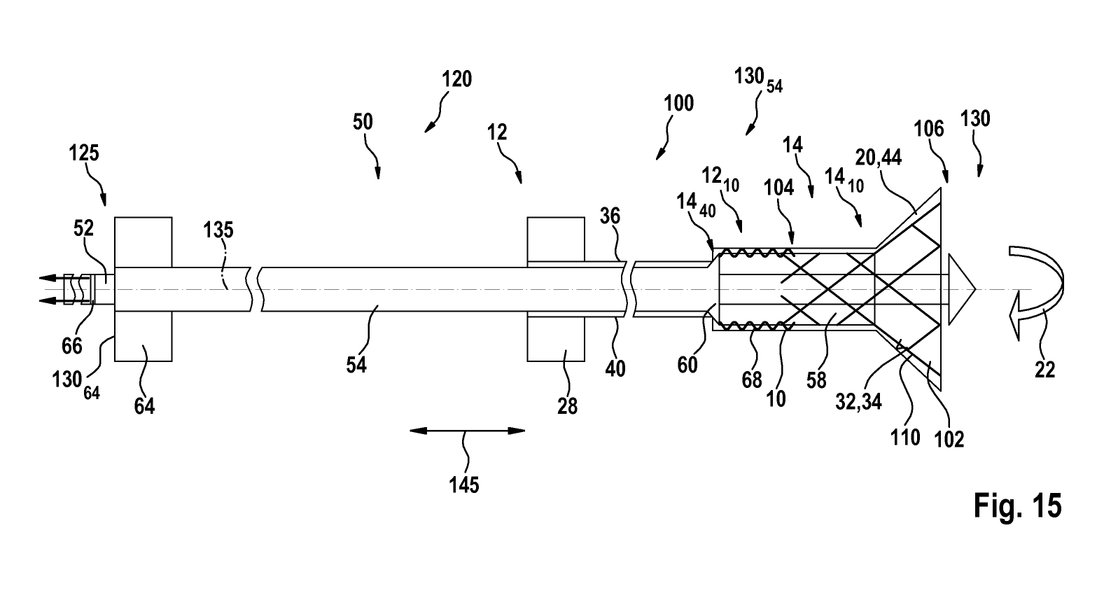

It would also be possible for the implant 102 in step 216 (resheathing) to be moved back incorrectly into the protective sleeve 58 by a movement of the first insertion element 52 in the axial direction 145 toward the proximal end 125 of the insertion device 120 (see arrow in FIG. 15). Alternatively, the implant 102 could also become stuck during a movement of the second insertion element 54 in the axial direction 145 toward the distal end 130 of the insertion device 120, whereby it may not be covered. In either case, a fold is thus formed in a region 68 of the protective sleeve 58, whereby resheathing is made impossible, as symbolized in FIG. 16 by a cross. In this case, the expansion body 10 can then help to form a sleeve around the implant 102 in step 214 (removal) as the insertion device 120 is withdrawn with the implant 102, the sleeve preventing injury to the patient during explantation.

In principle, one of the component parts/components of the insertion device 120 or of the implant 102 may also be formed from a metal that is visible under X-ray, such as stainless steel, tantalum, gold or platinum. An advance for example of the catheter tip 140 or of one of the insertion elements 52, 54 and therefore of the implant 102 as well as a correct position of the implant 102 at the site of implantation 150 could thus be monitored with the aid of an X-ray device (not shown here) during the implantation of the implant 102 by means of the insertion device 120.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### REFERENCE SIGNS

| | |
|---|---|
| 10 | expansion body |
| 12 | end |
| 14 | end |
| 16 | extension |
| 18 | extension |
| 20 | portion |
| 22 | circumferential direction |
| 24 | braid |
| 26 | round wire |
| 28 | grip segment |
| 30 | mesh |
| 32 | partial region |
| 34 | coating |
| 36 | stabilizing tube |
| 38 | extension |
| 40 | shaft casing |
| 42 | crossing point |
| 44 | third |
| 50 | shaft region |
| 52 | insertion element |
| 54 | insertion element |
| 56 | inner surface |
| 58 | protective sleeve |
| 60 | connection region |
| 62 | extension |
| 64 | grip segment |
| 66 | stop |
| 68 | region |
| 100 | cover device |
| 102 | implant |
| 104 | end |
| 106 | end |
| 108 | extension |
| 110 | outer contour |
| 120 | insertion device |
| 125 | end |
| 130 | end |
| 135 | inner axis |
| 140 | catheter tip |
| 145 | direction |
| 150 | site of implantation |
| 155 | aortic arch |
| 160 | aortic valve |
| 200 | pre-dilation |
| 202 | insertion |
| 204 | positioning |
| 206 | partial expansion |
| 208 | check |
| 210 | test |
| 212 | expansion |
| 214 | removal |
| 216 | resheathing |
| 218 | repositioning |
| 220 | examination |
| b | width |
| d | diameter |
| L | length |
| α | angle of crossing |

What is claimed is:

1. A cover device (100) for covering an at least partly released medical implant (102), which can be released from an insertion device (120) by a relative movement between a first and a second insertion element (52; 54), the cover device comprising at least one expansion body (10), at which a first radial extension (16) and at least one second radial extension (18) can be set, wherein the second radial extension (18) of the at least one expansion body (10) can be adapted to at least one radial extension (108) of the at least partly released medical implant (102) when the at least partly released medical implant (102) is covered, wherein the at least one expansion body (10) can be expanded substantially homogeneously in the circumferential direction (22) at least in at least one axial portion (20).

2. The cover device as claimed in claim 1, wherein the at least one expansion body (10), at least once the second radial extension (18) has been set, has a restoring force, which acts relative to an inner axis (135) of the insertion device (120) and which is designed to trigger a reversal of the expansion of the at least partly released medical implant (102).

3. The cover device as claimed in claim 2, wherein the at least second radial extension (18) of the at least one expansion body (10) is twice as large, optionally three times as large, and optionally more than five times as large, as the first radial extension (16) of the at least one expansion body (10).

4. The cover device as claimed in claim 1, wherein the at least one expansion body (10) has at least one braid (24), which is reversibly collapsible so as to set the first radial extension (12) and the at least second radial extension (18).

5. The cover device as claimed in claim 4, wherein the at least one expansion body (10) has at least one braid (24), which is fabricated from interwoven round wire (26).

6. The cover device as claimed in claim 1, wherein the at least one expansion body (10) is fabricated from Elgiloy.

7. The cover device as claimed in claim 1, comprising a proximal end (12), which is remote from a distal end (130) of the insertion device (120) during use, and a distal end (14), which faces the distal end (130) of the insertion device (120) during use, wherein the at least one expansion body (10) is arranged at the distal end (14) and/or at least one grip segment (28) is arranged at the proximal end (12).

8. The cover device as claimed in claim 1, wherein the at least one expansion body (10) has a proximal end (12₁₀) during use, which is remote from the distal end (130) of the insertion device (120) during use, and a distal end (14₁₀), which faces the distal, end (130) of the insertion device (120) during use, and wherein at least the distal end (14₁₀) of the at least one expansion body (10) is tapered and/or in particular is formed with closed meshes (30, 30′).

9. The cover device as claimed in claim 1, wherein the at least one expansion body (10) has a coating (34) at least in a partial region (34), and/or wherein the coating (34) is formed by a plastics film.

10. The cover device as claimed in claim 1, comprising a proximal end (12), which is remote from a distal end (130) of the insertion device (120) during use, and a distal end (14), which faces the distal end (130) of the insertion device (120) during use, wherein the proximal end (12) and the distal end (14) are fabricated from different materials.

11. The cover device at least as claimed in claim 1, designed as a stabilizing tube (36) of the insertion device (120).

12. An insertion device (120) for inserting a medical implant (102), which can be released by a relative movement between a first and a second insertion element (52; 54), the insertion device comprising a cover device (100) for covering a partly released medical implant (102), in particular as claimed in claim 1, the cover device comprising at least one expansion body (10), at which a first radial extension (16) and at least one second radial extension (18) can be set, wherein the second radial extension (18) of the at least one expansion body (10) can be adapted to at least one radial extension (108) of the at least partly released medical implant (102) when the at least partly released medical implant (102) is covered, wherein the at least one expansion body (10) can be expanded substantially homogeneously in the circumferential direction (22) at least in at least one axial portion (20).

13. The insertion device as claimed in claim 12, wherein the at least one expansion body (10) of the cover device (100) is placed radially around at least one of the insertion elements (52, 54) and/or is arranged axially movably with respect to at least one of the insertion elements (52, 54).

14. The insertion device as claimed in claim 12, wherein the implant (102) is designed as a self-expanding implant (102).

15. A method for covering an at least partly released implant (102) with a cover device (100) as claimed claim 1 of an insertion device (120), the method comprising at least the following steps:
implementing a relative movement at least of an expansion body (10) of the cover device (100) with respect to the at least partly released implant (102) and thus adapting at least one second radial extension (18) of the at least one expansion body (10) to at least one radial extension (108) of the at least partly released medical implant (102), wherein the at least one expansion body (10) is expanded substantially homogeneously in the circumferential direction (22) at least in at least one axial portion (20), and in particular
reversing the expansion of the at least partly released medical implant (102), triggered by a restoring force at least of the expansion body (10) of the cover device (100) acting relative to an inner axis (135) of the insertion device (120).
